(19) United States
(12) Patent Application Publication
(10) Pub. No.: US 2024/0358726 A1
(43) Pub. Date: Oct. 31, 2024

(54) SPECIFIC THERAPEUTIC MEDICAL MARIJUANA DOSES FOR STRESS AND PAIN

(71) Applicant: Yale University, New Haven, CT (US)

(72) Inventor: Rajita Sinha, New Haven, CT (US)

(73) Assignee: Yale University, New Haven, CT (US)

(21) Appl. No.: 18/688,040

(22) PCT Filed: Sep. 2, 2022
(86) PCT No.: PCT/US2022/042454

§ 371 Date: Feb. 29, 2024

Related U.S. Application Data
(60) Provisional application No. 63/240,702, filed on Sep. 3, 2021.

(51) Int. Cl. A61K 31/00

(52) U.S. Cl. CPC A61K 31/658

(57) ABSTRACT

In various aspects and embodiments the invention provides a method of treating pain, stress and anxiety in a subject in need thereof, wherein the method comprises administering to the subject in need thereof a pharmaceutical composition comprising cannabidiol (CBD) and tetrahydrocannabinol (THC) at a ratio between about 3:1 and about 6:1 CBD:THC.

### CROSS-REFERENCE TO RELATED APPLICATIONS

This application claims priority under 35 U.S.C. § 119(e) to U.S. Provisional Patent Application No. 63/240,702, filed Sep. 3, 2021, the disclosures of which is incorporated herein by reference in its entirety.

### BACKGROUND OF THE INVENTION

Medical use of cannabis products has expanded rapidly in recent years. Common reasons for use are alleviation of pain and also for reducing stress. However, little systematic and rigorous work has been done to maximize the efficacy of these products for use in the treatment of pain and management of stress. There is a need in the art for such compositions and methods.

The present disclosure addresses this need.

### SUMMARY OF THE INVENTION

In one aspect, the invention provides a method of treating pain in a subject in need thereof, the method comprising administering to the subject a pharmaceutical composition comprising cannabidiol (CBD) and tetrahydrocannabinol (THC) at a ratio between about 3:1 and about 6:1 CBD:THC.

In another aspect, the invention provides a method of treating anxiety in a subject in need thereof, the method comprising administering to the subject a pharmaceutical composition comprising cannabidiol (CBD) and tetrahydrocannabinol (THC) at a ratio between about 3:1 and about 6:1 CBD:THC.

In yet another aspect, the invention provides a pharmaceutical composition comprising cannabidiol (CBD) and tetrahydrocannabinol (THC) at a ratio of about 4:1 or about 3:1 CBD:THC.

In various embodiments, the ratio is about 4:1 or about 3:1 CBD:THC. In various embodiments, the pharmaceutical composition comprises 100 mg CBD+30 mg THC. In various embodiments, the pharmaceutical composition comprises 40 mg CBD+10 mg THC.

In various embodiments, the subject is a human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1B: Plasma levels of CBD and THC—adjusted for baseline, for each of the dose conditions over the course of 4.45 hours (285 minutes). FIG. 1A: Relative to placebo, plasma CBD levels significantly increased for the CBD100 (p<0.0005), CBD40+THC10 (p<0.01), and for CBD100+THC30 (p<0.0001) but not for CBD40 alone (p<0.34) or CBD40+THC20 (p<0.07). FIG. 1B: Relative to placebo, plasma THC levels significantly increased for the CBD40+THC20 (p<0.01) and for CBD100+THC30 (p<0.0001) but not for CBD40 alone (p<0.89) or CBD100 alone (p<0.76) or CBD40+THC10 (p<0.14).

FIGS. 2A-2D: Blood Pressure, Pulse and Wanting More Drug Rating, adjusted for Baseline. FIG. 2A: Relative to placebo, average systolic blood pressure (BP) adjusted for baseline was significantly decreased in the CBD40/THC10 (p<0.01) and the CBD100/THC30 doses (p<0.001), but not for other doses. FIG. 2B: Relative to placebo, diastolic BP adjusted for baseline was significantly increased for the CBD100 dose (p<0.05) but not for any other dose. FIG. 2C: Relative to placebo, average pulse adjusted for baseline decreased for all doses non-significantly, and was significantly increased by an average of 2 bpm for the CBD100+THC30 dose (p<0.001). FIG. 2D: Relative to placebo, average drug craving (want more drug) was significantly reduced for all doses including CBD40 (p<0.001), CBD100 (p<0.001), CBD40+THC10 (p<0.05), CBD40+THC20 (p<0.01) and CBD100+THC30 (p<0.001).

FIGS. 3A-3B: Provoked average pain and anxiety (adjusted for baseline) ratings during adapted CPT protocol. FIG. 3A: Relative to placebo, average provoked pain ratings were significant decreased in the CBD40/THC10 and in the CBD10/THC30 doses (p's<0.05), but not for other doses. FIG. 3B: Relative to placebo, average adjusted anxiety ratings were only significant reduced in the CBD100/THC30 dose (p<0.01) and not for any other dose.

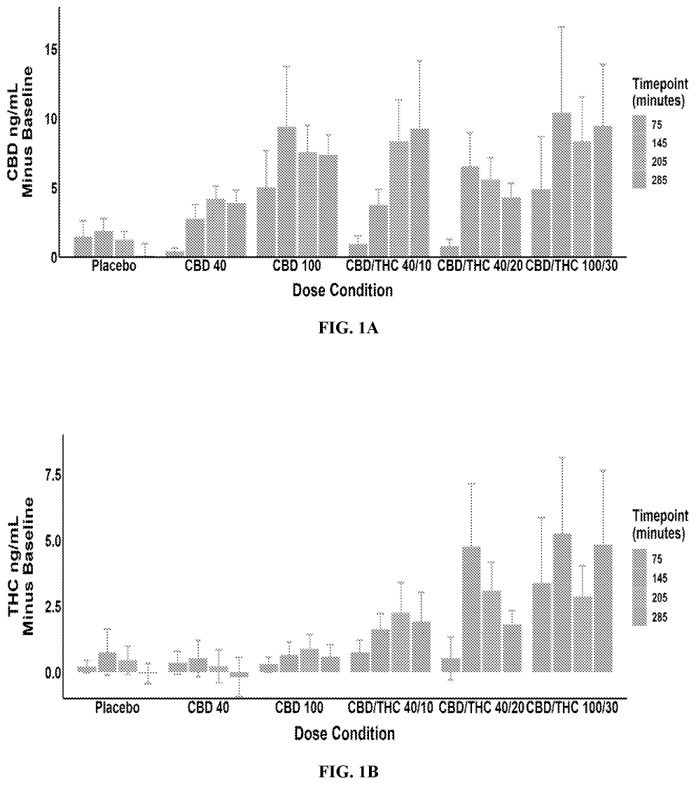

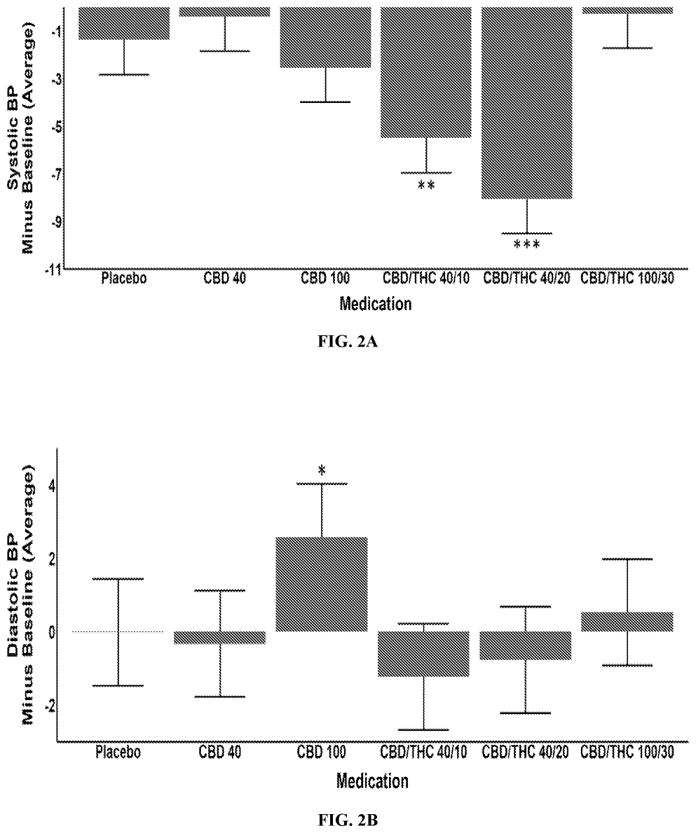

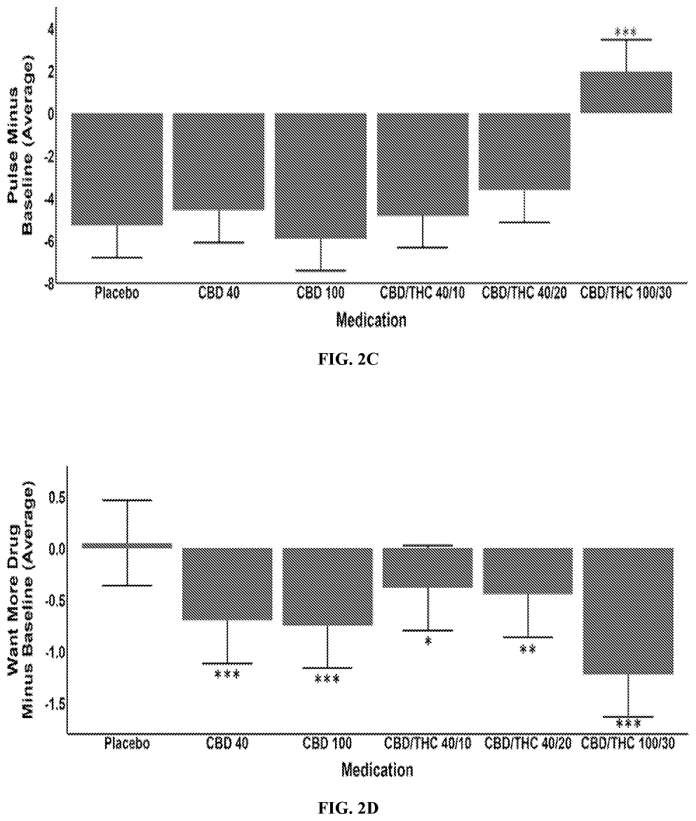

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

As used herein, the term “CBD” or “cannabidiol” refers to the molecule having the formula:

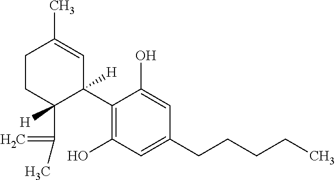 [SMILES: C=C(C)[C@@H]1CCC(C)=C[C@H]1c1c(O)cc(CCCCC)cc1O]

and pharmaceutically acceptable salts thereof.

As used herein, the term “THC” or “tetrahydrocannabinol refers to the molecule having the formula:

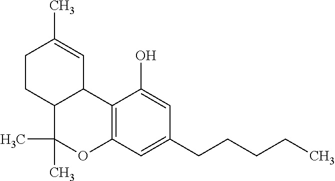 [SMILES: CCCCCc1cc(O)c2c(c1)OC(C)(C)C1CCC(C)=CC21]

and pharmaceutically acceptable salts thereof.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly, the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

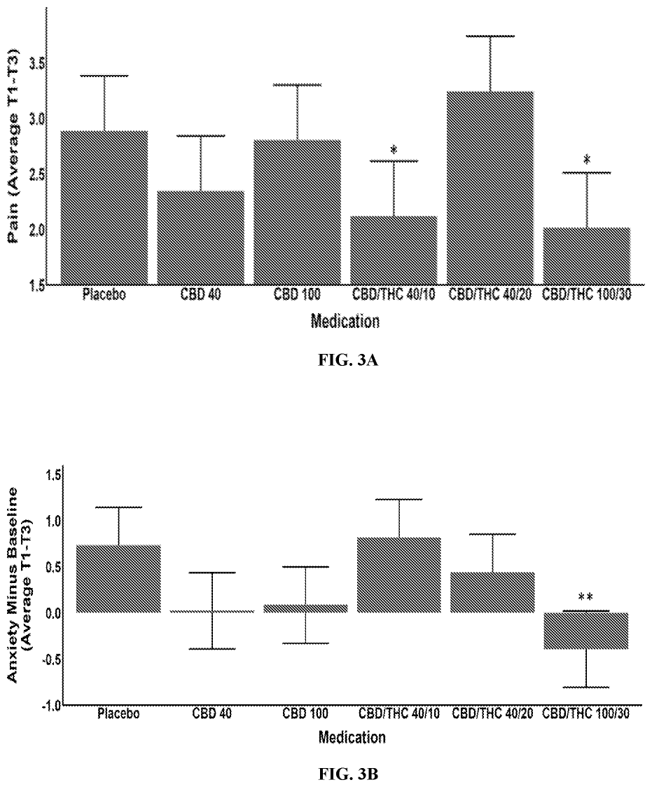

In one aspect, the invention provides a method of treating pain in a subject in need thereof, the method comprising administering to the subject a pharmaceutical composition comprising cannabidiol (CBD) and tetrahydrocannabinol (THC) at a ratio between about 3:1 and about 6:1 CBD:THC. In various embodiments, the ratio is about 4:1 or about 3:1 CBD:THC. In another aspect, the invention provides a method of treating anxiety in a subject in need thereof, the method comprising administering to the subject a pharmaceutical composition comprising cannabidiol (CBD) and tetrahydrocannabinol (THC) at a ratio of about 4:1 or about 3:1 CBD:THC. Without meaning to be limited by theory, compositions comprising specific ratios of CBD to THC show heightened efficacy in the treatment of pain (FIG. 3A) and anxiety (FIG. 3B) relative to compositions comprising other ratios of these compounds.

In various embodiments, the pharmaceutical composition comprises 100 mg CBD+30 mg THC. In various embodiments, the pharmaceutical composition comprises 40 mg CBD+10 mg THC.

In various embodiments, subject is a mammal. In various embodiments, the subject is a human. A skilled person will recognize that a wide variety of dosage forms may be employed in various aspects and embodiments of the methods of the invention. In various embodiments the pharmaceutical composition is configured to be delivered as a nasal spray, orally, subcutaneously, parenterally, sublingually, as a lozenge or as a solid or liquid formulation.

In another aspect, the invention provides a pharmaceutical composition comprising cannabidiol (CBD) and tetrahydrocannabinol (THC) at a ratio of about 4:1 or about 3:1 CBD:THC. In various embodiments, the pharmaceutical composition comprises at least one pharmaceutically acceptable excipient.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

Eight 21-45-year-old men and women, who are recreational marijuana users but do not meet criteria for cannabis use disorder were recruited to complete six separate inpatient or outpatient laboratory sessions one week apart during which they were assigned to receive an acute dose of either 40 mg CBD, 100 mg CBD, 40/10 mg each of CBD/THC, 40/20 mg of CBD/THC and 100/30 mg each CBD/THC or PLA, in a random, counter-balanced, double-blind, cross-over design over a 7-week period. Sessions were completed one-week apart to allow for an adequate washout period between sessions.

The first study was directed towards assessing safety of the THC and CBD doses in healthy, recreational cannabis users. The key novel and surprising aspect of this study was the specific combinations of CBD+THC that were found to be significant in reducing pain and stress responses. Surprisingly, CBD alone (often thought of as pain reducing) was not as effective as a combination of CBD with low doses of THC in the 4:1 and 3:1 range but not in the 2:1 proportion range. So 100 CBD+30 THC and the 40 CBD+10 THC were significantly different that then 40 CBD+20 THC and the 100 CBD alone and 40 CBD alone. This suggests that low amounts of THC may act synergistically to increase therapeutic efficacy for pain, but not at moderate (2:1) or higher amounts (1:1). The novel pain provocation experimental method allowed for detection of this effect within this first safety protocol assessment.

### Example 2

Study 2: Arm 1: Adult men and women ages 21-68 (N=12) with chronic pain (not fully controlled by opioid pain medication), will be recruited and randomly assigned to receive the two doses of CBD40+THC10 (mg) and CBD100+THC30 (mg) dose (n=4 in each) or PLA (n=4) repeated dosing for 7 days. On day 1 and 6/7 of the 7-day dosing, subjects will complete laboratory sessions. Subjects cannot meet criteria for moderate/severe cannabis use disorder or other addictive disorders.

Arm 2: Adult men and women ages 21-68 (N=12) with chronic pain and Opioid Use Disorder (OUD) (not fully controlled by opioid pain medication), will be recruited and randomly assigned to receive the two doses of CBD40+THC10 (mg) and CBD100+THC30 (mg) dose (n=4 in each) or PLA (n=4) repeated dosing for 7 days. On day 1 and Day 6/7 of the 7-day dosing, subjects will complete laboratory sessions. Subjects cannot meet criteria for moderate/severe other addictive disorders.

Arm 3: Adult men and women ages 21-68 (N=12) with chronic pain and Multiple Sclerosis (and pain not fully controlled by opioid pain medication), will be recruited and randomly assigned to receive the two doses of CBD40+THC10 and CBD100+THC30 dose (n=4 in each) or PLA (n=4) repeated dosing for 7 days. On day 1 and Day 6/7 of the 7-day dosing, subjects will complete laboratory sessions. Subjects cannot meet criteria for moderate/severe cannabis use disorder or other addictive disorders.

In each case the combination of CBD and THC at the ratios taught herein will exhibit greater efficacy than these compounds administered alone or at other ratios.

### Enumerated Embodiments

The following exemplary embodiments are provided, the numbering of which is not to be construed as designating levels.

Embodiment 1 provides a method of treating pain in a subject in need thereof, the method comprising administering to the subject a pharmaceutical composition comprising cannabidiol (CBD) and tetrahydrocannabinol (THC) at a ratio between about 3:1 and about 6:1 CBD:THC.

Embodiment 2 provides the method of embodiment 1, wherein the ratio is about 4:1 or about 3:1 CBD:THC.

Embodiment 3 provides the method of embodiments 1-2, wherein the pharmaceutical composition comprises 100 mg CBD+30 mg THC.

Embodiment 4 provides the method of embodiments 1-3, wherein the pharmaceutical composition comprises 40 mg CBD+10 mg THC.

Embodiment 5 provides the method of embodiments 1-4, wherein the subject is a human.

Embodiment 6 provides a method of treating anxiety in a subject in need thereof, the method comprising administering to the subject a pharmaceutical composition comprising cannabidiol (CBD) and tetrahydrocannabinol (THC) at a ratio between about 3:1 and about 6:1 CBD:THC.

Embodiment 7 provides a method of embodiment 6, wherein the ratio is about 4:1 or about 3:1 CBD:THC.

Embodiment 8 provides a method of embodiments 6-7, wherein the pharmaceutical composition comprises 100 mg CBD+30 mg THC.

Embodiment 9 provides a method of embodiments 6-8, wherein the pharmaceutical composition comprises 40 mg CBD+10 mg THC.

Embodiment 10 provides a pharmaceutical composition comprising cannabidiol (CBD) and tetrahydrocannabinol (THC) at a ratio of about 4:1 or about 3:1 CBD:THC.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating pain in a subject in need thereof, the method comprising administering to the subject a pharmaceutical composition comprising cannabidiol (CBD) and tetrahydrocannabinol (THC) at a ratio between about 3:1 and about 6:1 CBD:THC.

2. The method according to claim 1, wherein the ratio is about 4:1 or about 3:1 CBD:THC.

3. The method according to claim 1, wherein the pharmaceutical composition comprises 100 mg CBD+30 mg THC.

4. The method according to claim 1, wherein the pharmaceutical composition comprises 40 mg CBD+10 mg THC.

5. The method according to claim 1, wherein the subject is a human.

6. A method of treating anxiety in a subject in need thereof, the method comprising administering to the subject a pharmaceutical composition comprising cannabidiol (CBD) and tetrahydrocannabinol (THC) at a ratio between about 3:1 and about 6:1 CBD:THC.

7. The method according to claim 6, wherein the ratio is about 4:1 or about 3:1 CBD:THC.

8. The method according to claim 6, wherein the pharmaceutical composition comprises 100 mg CBD+30 mg THC.

9. The method according to claim 6, wherein the pharmaceutical composition comprises 40 mg CBD+10 mg THC.

10. A pharmaceutical composition comprising cannabidiol (CBD) and tetrahydrocannabinol (THC) at a ratio of about 4:1 or about 3:1 CBD:THC.
